(19) United States
(12) Patent Application Publication
(10) Pub. No.: US 2007/0197888 A1
(43) Pub. Date: Aug. 23, 2007

(54) Blood Oxygenation Sensor

(75) Inventors: Noel Axelrod, Bnei Brak (IL); Eran Ofek, Bnei Brak, IL (US)

Correspondence Address: EDWARD S. SHERMAN, ESQ., 3554 ROUND BARN BLVD., SUITE 303, SANTA ROSA, CA, 95403, US

(73) Assignee: PHYSICAL LOGIC AG, Zug (CH)

(21) Appl. No.: 11/676,301

(22) Filed: Feb. 18, 2007

Related U.S. Application Data
(60) Provisional application No. 60/775,531, filed on Feb. 21, 2006.

(51) Int. Cl. A61B 5/00

(52) U.S. Cl. 600/339

(57) ABSTRACT

A planar dielectric waveguide sensor is used to determine the concentrations of oxygenated and deoxygenated hemoglobin and other blood constituents such as pH, sugar. The planar waveguide core is in direct contact with the blood such that evanescent field of the light propagating within the core is selectively attenuated at specific wavelengths of interest. The planar waveguide has a construction that promotes strong interaction of the evanescent field with blood cells that contact it. In preferred embodiments, the waveguide is constructed of a low refractive index core to propagate an evanescent wave comparable in size to a red blood cell.

### CROSS REFERENCE TO RELATED APPLICATIONS

The present application claims priority to the U.S. Provisional Patent Application for a “Blood Oxygenation sensor, filed on Feb. 21, 2006, and now assigned application Ser. No. 60/775,531, which is incorporated herein by reference.

### BACKGROUND OF INVENTION

The present invention relates to methods and an apparatus for the in-vivo optical measurement of blood chemistry, and in particular the oxygenation of red blood cells.

Prior art methods of measuring the oxygenation of blood are well known and utilize various means to measure light absorption at specific wavelengths in the Near Infrared (NIR) of the spectrum of electromagnetic radiation to distinguish between the concentration of the oxygenated hemoglobin and de-oxygenated or reduced hemoglobin in red blood cells (RBC's). Externally worn sensors, typically deployed on a finger of other thin external appendage, are widely deployed but give a gross average of the oxygenation of RBC's in the large number of veins and artery that are illuminated by an external light source. Of more interest to researchers and clinicians are local arterial measurements at a specific location.

U.S. Pat. No. 5,280,786 to Wlodarczyk et al. issued on Jan. 25, 1994 for an Fiberoptic blood pressure and oxygenation sensor deployed on a catheter placed transcutaneously into a blood vessel. A sensing tip of the catheter includes a pressure-sensing element and an oxygen saturation-measuring element. One of the disclosed means for measuring the oxygen saturation of blood was a sensing tip comprising an optical fiber having at least a portion of the cladding removed. Other features of this invention included novel tip designs, measuring head features, and approaches for enhancing measurement though correlation of the saturation and pressure readings.

While the device disclosed in the '786 patent was an improvement of the prior art in providing a means to measure oxygenation in a single vein or artery it appears to still suffer several limitations of potential consequence to more extensive an long term clinical deployment in patents.

One limitation recognized by the inventors in the current invention is that the geometry created by the removal of the fiber cladding and to enable the other features is that it creates an irregular protruding device in the blood stream. As such, a device is placed in smaller arteries and veins, where the flow velocity is higher; there is a greater likelihood that the protrusion will result in turbulent blood flow that can disrupt RBC's leading to hemolysis.

For such reasons, and others it would be desirable to have a fiber optic sensor such as that disclosed in the '786 patent that is at least one of more sensitive and smaller in size, and preferably both and does not suffer from the disadvantages of prior art.

It is therefore a first object of the present invention to provide an optical means for precise local measurement of oxygenated hemoglobin and reduced hemoglobin that can potentially be smaller than the size provided by the prior art.

It is a further objective of the present invention to provide an optical sensing means that is sufficiently compatible with blood that it can remain in a patient for a long period of time, and be deployed in smaller veins and/or arteries.

It is yet another object of the present invention to provide such a sensor device that is easier to integrate with other biomedical devices and transducers

It is also a further objective of the present invention to provide such a device that is capable of a far more accurate local and representative determination of concentrations of in-vivo blood components, including oxygenated hemoglobin and reduced hemoglobin.

### SUMMARY OF INVENTION

In the present invention, the first object of having a reduced size device that is non-protruding is achieved by providing in optical communication a laser or other light source, a beam splitter and a first detector to receive a portion of the energy directed to it by the beam splitter. The other portion of the energy directed by a beam splitter is transported to a planar waveguide in contact with the blood. Light from the laser or other source may be delivered to the planar waveguide by an optical fiber. The light attenuated by absorption of the evanescent wave in the planar waveguide is directed to a second detector. When the light is delivered by an optical fiber, the light direction means is preferably a mirror at the end of the optical fiber.

A second object of the invention of providing higher sensitivity and more representative measurement of the local concentration of oxygenated and reduced hemoglobin is achieved by providing either a planar or an optical fiber waveguide sensing device in contact with the blood wherein the refractive index of the core of the waveguide is preferably less than about 1.47. The refractive index of the waveguide core exposed to the blood is more preferably between about 1.38-1.42, the refractive index of the human RBC's, to allow the evanescent light to penetrate deeply into more RBC's than prior art devices.

The above and other objects, effects, features, and advantages of the present invention will become more apparent from the following description of the embodiments thereof taken in conjunction with the accompanying drawings.

### BRIEF DESCRIPTION OF DRAWINGS

FIG. 1A a schematic illustration of the planar dielectric waveguide with the sandwiched between two cladding regions in which the guided waves propagate in the z direction.

FIG. 1B illustrates the electric field distribution 15 within single mode dielectric waveguide 10 of FIG. 1A and as it spreads into the cladding 11 where its strength is exponentially decaying in intensity.

FIG. 2 is a graphic representation showing the dependence of the imaginary part of the effective refractive index n_{eff}″ on the imaginary part of the cladding refractive index n₂″.

FIG. 3 is a graphic representation of showing the dependence of γ′, the exponential decay factor in the cladding from equation 9, on the imaginary part of the cladding refractive index n₂″.

FIG. 4 shows the absorption spectrum of water in the 600-1100 nm range.

FIG. 5 compares the absorption spectra of oxygenated and reduced hemoglobin in the ranges 450-1000 nm (top), and 650-1050 nm (bottom) as published by Cope 1991 (M. Cope. Ph.D. Thesis, Univ. College London, 1991)

FIG. 6A is a schematic illustration of the device deployed in the method for measuring concentrations of Hb and HbO₂.

FIG. 6B is a cross sectional elevation through the schematic oximetry probe portion of the device in FIG. 6A.

FIG. 6C is a cross section elevation of the oximetry probe portion of the device in FIGS. 6A and 6B showing the optical fiber, coupler and planar waveguide at section line C-C in FIG. 6B

FIG. 7 illustrate the dependence of β″ on saturation level SO₂

FIG. 8 illustrates the power extinction (Eq. 16) as a function of the saturation level in a Log-Linear scale respectively to better show that the extinction increases logarithmically with the saturation level

FIG. 9 illustrates in perspective the geometry of the dielectric waveguide 130 used for measuring oxygen saturation level of blood.

FIG. 10 illustrates the single mode dielectric waveguide electric field distribution for the dielectric waveguide 130 of FIG. 9

FIG. 11 is a graph illustrating the refractive index of hemoglobin and water in the range 250-1050 nm.

FIG. 12 compares the dispersion of the refractive index of the HOSP in the infrared, visible and ultraviolet ranges with that of SiO2. Note that the wavelength is indirectly shown as the energy in eV.

FIG. 13A is an illustration of the geometry of the planar dielectric waveguide consisting of the core 130 and cladding 135 as surrounded by water 5 in which a single red blood cell particle 200 placed on the waveguide surface.

FIG. 13B illustrates the results of modeling the electric field intensity distribution for the geometry shown in FIG. 13A. The field propagates from the left to the right of the waveguide 130.

### DETAILED DESCRIPTION

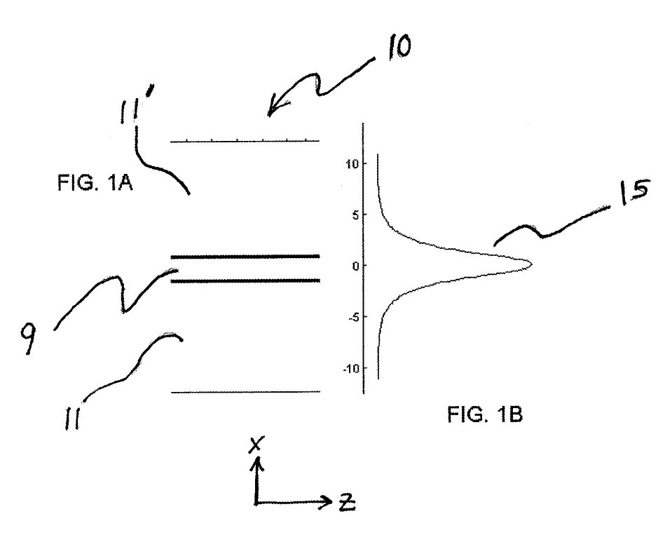

Referring to FIGS. 1 through 13, wherein like reference numerals refer to like components in the various views, there is illustrated therein a new and improved blood oxygenation sensor, generally denominated 100 herein.

Before providing details on the construction and methods of using the various embodiments of the invention, theoretical consideration regarding the operative principles of such device will be described.

A step index optical fiber/dielectric waveguide has, basically, two regions with different refractive indexes: core and cladding. The core has a refractive index larger than the cladding region surrounding it. Light waves are guided throughout the waveguide due to a phenomenon known as total internal reflection. Remarkably, that propagating field is not spatially limited only to the core, but also extends into the cladding and exponentially decays with the distance from the core. This exponentially decaying field is known as an evanescent field. The extension of the evanescent field depends on the core diameter and the refractive indexes of the core and the cladding.

The absorption coefficient of silicate glass is very small in the visible and infrared ranges of the spectrum; because of that, the light absorption in the waveguide is usually neglected in the analysis of guiding modes. In contrast to the textbook analysis, in the treatment below, we consider a waveguide with a transparent core and an absorbing cladding media.

The propagation of light within dielectric medium is governed by the Helmholtz equation:
ΔE+n²k₀²E=0 (1)
where E=E(x, y, z) is the electric field, n is the refractive index and k₀=ω/c=2π/λ is a free-space light wavevector.

The Helmholtz equation remains valid also in the case of an absorbing medium. In that case the refractive index is a complex quantity:
n=n′+in″ (2)
where n″ describes light absorption:
α=2k₀n″ (3)
where α is the absorption coefficient.

For the sake of simplicity, we consider only a symmetric planar dielectric waveguide 10 as schematically illustrated in FIG. 1A. The planar dielectric waveguide core 9 is sandwiched between two cladding regions 11′ and 11 in which the guided waves propagate in the z direction.

We consider only the case of the transverse electric field. The case of the transverse magnetic field could be treated in the analogously. Seeking the solution of the Eq. (1) in the form E(x, y, z)=ŷE_{y}(x)exp(iβz), we obtain the following equations for the propagating field

$$\frac{{\partial }^{2} {E}_{y}}{\partial {x}^{2}} + ( {n}_{j}^{2} {k}_{0}^{2} - {β}^{2} ) {E}_{y} = 0 , \qquad ( 4 )$$

where β is the propagation constant, nⱼ, j={1, 2} is the refractive indexes of the core and the cladding respectively. Since the refractive index of cladding is a complex quantity the same is also true for β:
β=β′+iβ″. (5)

The imaginary part of β is responsible for decaying of the propagating wave. The intensity of the wave after propagation of a distance L is:
I=I₀ exp(−2β″L), (6)
where I₀ is the input intensity. This intensity could be measured by a photodetector. Our goal is to relate β″ with the absorption coefficient of the cladding medium α=2k₀n₂″. Alternatively, defining the effective refractive index n_{eff} as
β=k₀n_{eff}, (7)
the above problem could be reformulated as finding a relation between n₂″ and n_{eff}″. The general solution of the Eqs. (2) has the form
E_{y1}=A exp(ik₁x)+B exp(−ik₁x)
E_{y2}=C exp(−γx) (8)
where E_{y1}, E_{y2} are the fields in the core and the cladding respectively and k₁ and γ are the complex numbers given by
k₁=\sqrt{n₁²k₀²−β²}
γ=\sqrt{β²−n₂²k₀²} (9)

E_{y2} is the evanescent field. It extends into the cladding on the effective distance 1/Re[γ]. The constants k₁ and γ are calculated from continuity conditions of the field and its derivatives on the interface. Specifically, for the even modes: E_{y}(x)=E_{y}(−x), the following equation holds:
kₜ tan(k₁a)=γ (10)
wherein for the odd modes: E_{y}(x)=−E_{y}(−x) the following equation holds:
k₁ cot(k₁a)=−γ (11)
where a =d/2 is the half of the core thickness. From Eqs (9) follows:
k₁²+γ²=k₀²NA², (12)
where NA is the numerical aperture of the waveguide NA=\sqrt{n₁²−n₂²}. The transcendental equations Eqs. (10)-(11) together with Eq. 12 solve the above problem. The complex propagation constant β is calculated from Eqs (9):
β=\sqrt{n₁²k₀²−k₁²}=\sqrt{n₂²k₀²+γ²} (13)
If there is no absorption, then all quantities in Eqs. (9)-(13) are real numbers. In that case, the above equations coincide with the classical equations for modal dispersion in a planar symmetric dielectric waveguide. However, when the propagating medium is absorbing, the same equations solve the problem of finding dependence of the mode intensity decay rate 2β″ on the absorption coefficient of the cladding medium α.

FIG. 1B shows a single mode field intensity 15 in a non-absorbing dielectric waveguide 10 with core 9 and cladding layers 11′ and 11 above and below respectively. The field was calculated from Eqs. (7), (9) and (10) for a waveguide with a core thickness of d=2 μm, a light wavelength of λ=640 nm, a numerical aperture NA=0.11 and a core refractive index 1.45 (silica glass).

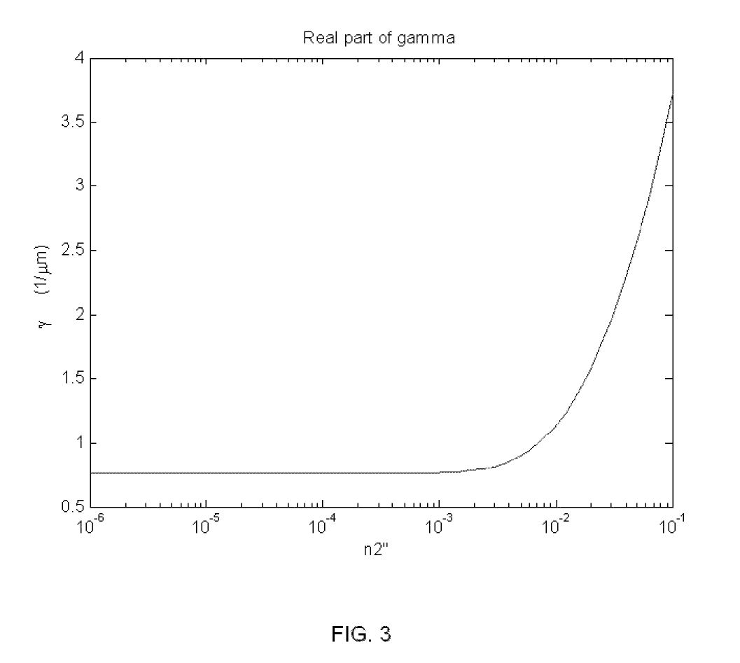

The dependence of the decaying rate 2β″=2k₀n_{eff}″ inside the dielectric waveguide in the example above on the absorption coefficient of cladding medium α=2k₀n₂″ is shown on the FIG. 3.

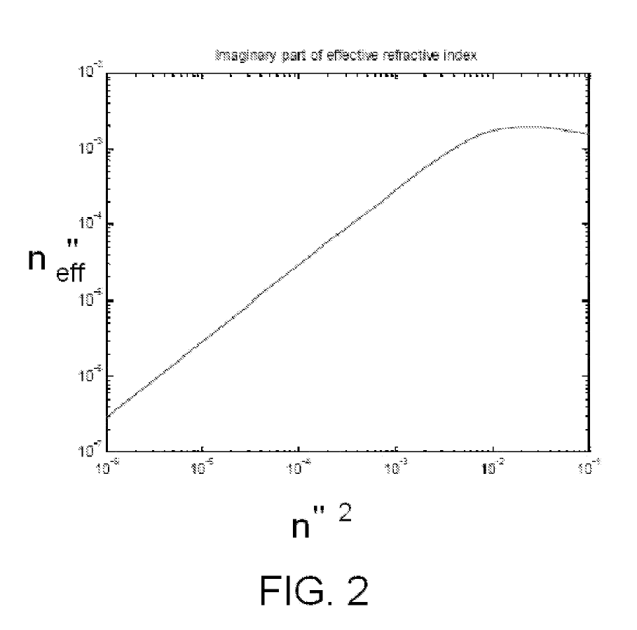

FIG. 2 shows, that for the values of n₂″ up to approximately 0.005 or, equivalently, up to absorption coefficient α=2k₀n₂″=0.1 μm⁻¹ the effective refractive index is approximately proportional to n₂″. The maximum of n_{eff}″ is at n₂″=0.02; after that value the decay rate decreases.

The dependence of γ′ on n₂″ is shown on the FIG. 3. As seen from the plot γ′ is remains constant up to n₂″≈10⁻³, after that γ′ increases rapidly. That corresponds to the decrease of the penetration depth of the evanescent field (which is 1/γ′) in a strongly absorbing medium.

The main constituents of blood which contribute towards absorption in visible and near infrared ranges are water and hemoglobin. While the former is constant, the concentrations of oxygenated hemoglobin (HbO₂) and reduced hemoglobin (Hb) change. Thus, the corresponding changes in absorption can provide clinically useful physiological information.

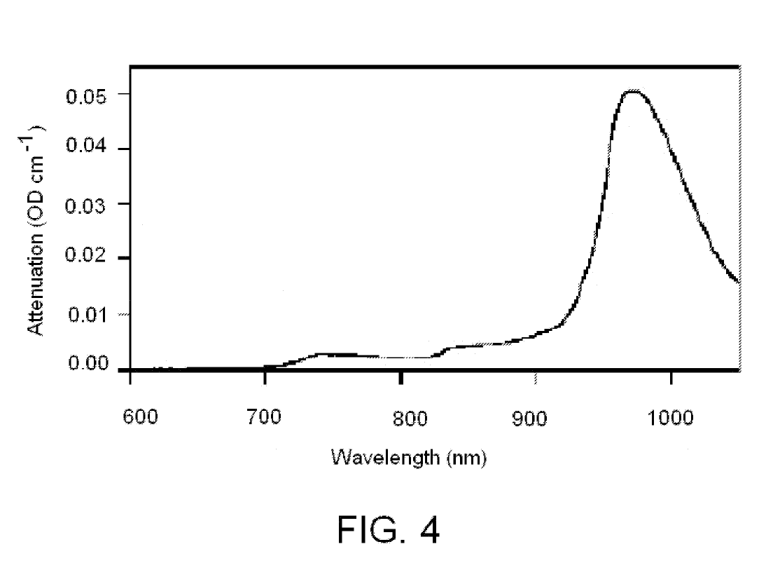

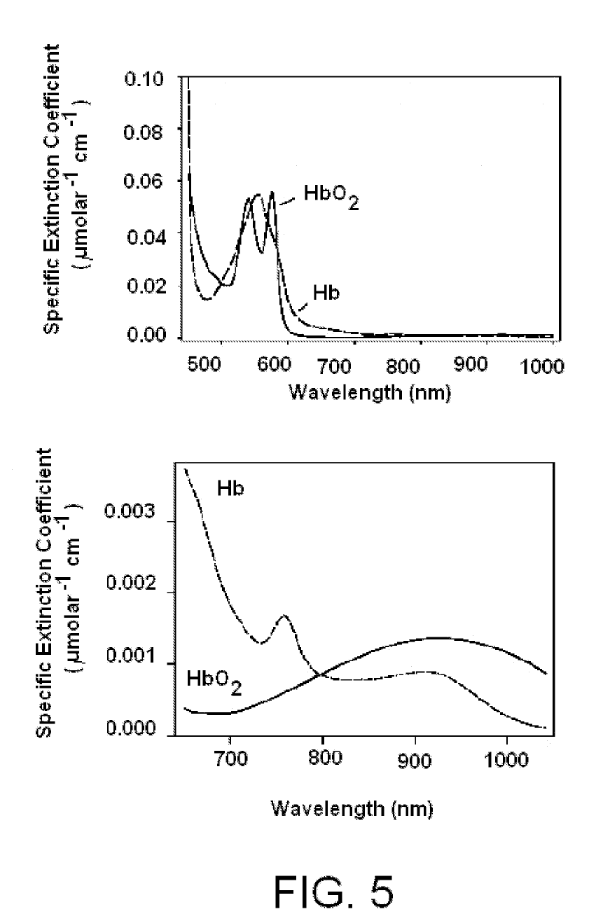

FIG. 4 shows absorption spectrum of water in the 600-1100 nm range. The significant absorption occurs only in the infrared region. The spectra of Hb and HbO2, expressed in term of the specific extinction coefficient, can be seen in FIG. 5. While both absorb strongly in the blue and green regions of the visible spectrum; the absorption of Hb is slightly stronger beyond about 590 nm. Note the point at about 800 nm, where the two curves intersect. The specific extinction coefficient μ represents the level of absorption per mmol of compound per liter of solution per cm (usually quoted in unit's mmolar-1 cm-1). It is related to the absorption coefficient α as:

$$μ = k \frac{α}{c} \qquad ( 14 )$$

where k=log₁₀(e)=0.4343 is a constant and c is the concentration of the compound (in units of mmoles).

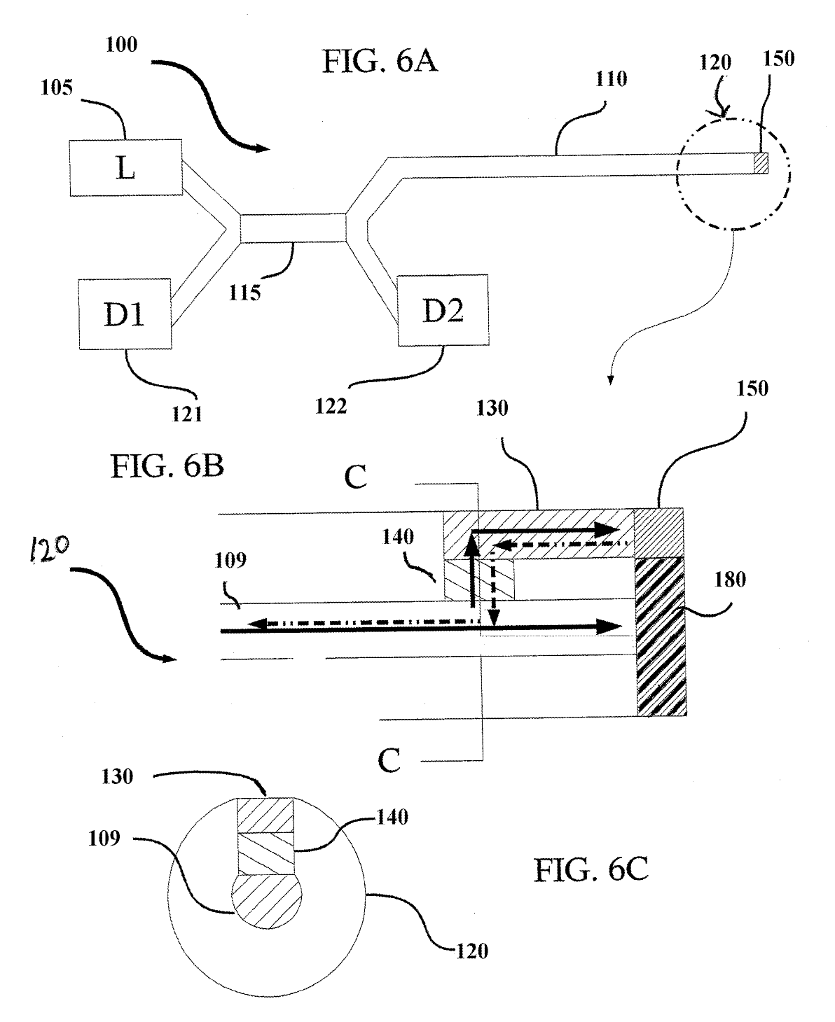

In accordance with a first embodiment of the invention, an oximeter device 100 for measuring concentrations of oxygenated or oxyhemoglobin (HbO₂) and reduced hemoglobin (Hb) is shown on FIG. 6, which shows that the various useful absorption bands of Hb and HbO₂ occur at wavelengths between about 550 nm and about 800 nm.

Light from a laser source 105 is coupled into a 50/50 2×2 fiber optic beamsplitter 115. Half of the light energy is coupled into an optical fiber 110 that ends with the oximetry probe 120 while the other half is measured on the detector D2 (122) which measures a reference signal.

In the optical fiber 110 the light propagates without losses until it enters the planar waveguide portion 130 exposed to the blood. Light enters planar waveguide portion 130 from fiber core 109 via a coupler 140, as shown by the solid arrows. In this region, the intensity of the wave exponentially decreases with the propagating distance due to interaction of the wave with the absorbing medium (blood) via its evanescent field. When the waves reach the end of the planar waveguide 130 or the fiber 120 they are reflected from the mirror on the fiber's end, as shown by the dashed arrows. An optical fiber 110 is the preferred means for delivery light from the laser source 105, as it can be readily adapted to fit into or form a catheter that is inserted into the body, and in particular the cardiovascular system. As an alternative to using coupler 140 to direct a portion of the light from optical fiber 110 to planar waveguide 130, optical fiber 110 may simply terminate in a planar waveguide having a mirror on the end face, or other means to return light back in the direction of the optical fiber indicated by the dashed arrows. It is also preferable that the light transmitted through the internal fiber but not coupled into the planar waveguide should be absorbed, rather than reflected by the mirror 150. Thus, it is more preferable that only the planar waveguide terminates in a mirror, with the end of the fiber optic terminating in an absorbing layer 180 or alternative structure or optical path that does not allow uncoupled light to reflect back to the detector.

Although the preferred embodiment deploys a mirror at the end of the fiber optic 110 used to deliver light to the planar waveguide 130, alternative embodiments include using a continuous optical fiber in the form of a loop that terminates at detector D2 (122) wherein a second optical coupler would transmit light from the planar waveguide in the same direction as propagation such that it reaches detector D2.

It should also be appreciated that rather than using a laser, a multi-wavelength light source might be deployed such as a broadband light source of multiple fiber optic lasers each tuned to a different wavelength.

The backreflected wave is transmitted through the exposed region and finally, after splitting on the beamsplitter 115 reaches the detector D1 (121). The light intensity decay in one pass of the exposed region is:
I=I₀ exp(−2β″ L), (15)
where β″ is the imaginary part of β in the Eq. (13) and L is the length of the exposed region. If the transmission losses of the optical fiber and losses on the mirror and beamsplitter are negligible in comparison with the losses in the exposed region, then the following equation holds:
exp(−4β″ L)=2I₁/I₂ (16)
where I₁, I₂ are the signals on detectors D1 and D2 respectively. The factor 4 in the exponent in Eq.(16) appeared because the double pass of the exposed region. From Eq. (16) the characteristic distance L₀ of the exposed region is:

$${L}_{0} = \frac{1}{4 {β}^{″}} . \qquad ( 17 )$$

At this distance the intensity decreases e times in a double pass. Although a more precise model should account for reflections at the interfaces between the cladding of the optical fiber and absorbing medium (blood) as well the losses due to fiber mode transformation when the mode field enters into the exposed region, we expect comparable trends to those shown herein.

The β″ can be expressed through the absorption coefficient of blood α by the solution of the modal dispersion equations.

The absorption coefficient of the blood depends on the concentrations of oxygenated and deoxygenated hemoglobin in it. The total absorption coefficient in the blood is a sum of specific absorption coefficients:
α=α₀+α_{Hb}+α_{HbO}_{₂} (18)
where α_{HbO}_{₂}, α_{Hb} and is the absorption coefficients of oxygenated and deoxygenated hemoglobin and α₀ is the absorption coefficient of blood without hemoglobin. α₀ is assumed to be constant on a short time scale on which α_{HbO}_{₂}, α_{Hb} changes. Using Eq. (14), Eq. (18) could be rewritten in the form:

$$α = {α}_{0} + \frac{{μ}_{Hb}}{k} {c}_{Hb} + \frac{{μ}_{Hb {O}_{2}}}{k} {C}_{Hb {O}_{2}} \qquad ( 19 )$$

where c_{Hb}, c_{HbO}_{₂} are concentrations of the Hb and HbO₂ respectively. Note that the absorption coefficient and specific extinction coefficients are functions of λ. If a measurement is done at two or more wavelengths of light, then we have the system of linear equations

$$α ( {λ}_{i} ) = {α}_{0} ( {λ}_{i} ) + \frac{{μ}_{Hb} ( {λ}_{i} )}{k} {c}_{Hb} + \frac{{μ}_{Hb {O}_{2}} ( {λ}_{i} )}{k} {C}_{Hb {O}_{2}} \qquad ( 20 )$$

where λᵢ {i=1, . . . , N} are wavelengths of the light. If the function α₀(λ) is known, then the system of Eqs. (20) could be solved for concentrations of the Hb and HbO₂. This system is over determined for N>2; it has a single solution at N=2 if the determinate of the system is not zero. If N=1, then the system is under determined and could not be solved. However, if the total concentration of hemoglobin c in blood is known, then using the additional equation:
c_{Hb}+c_{HbO}_{₂}=c, (21)

The concentrations c_{Hb}, c_{HbO}_{₂} could be determined as well for the case N=1.

To better appreciate certain aspects and embodiments of the present invention we first illustrate the optimum and effective operative parameter for measuring blood oxygenation with a fiber optic sensor operating at the single wavelength λ=600 nm. That is, rather than utilizing the coupler and dielectric waveguide shown in FIG. 6, we consider the performance when the oximeter probe is simply the terminal portion of the optical fiber when the cladding is removed or reduced in thickness such that an evanescent field might extend into the blood. As in a conventional optical fiber, the refractive index of the fiber core is n₁=1.45 and the core diameter is 2 μm. Blood is modeled as a medium with the refractive index n₂=1.33+iα/2k₀, where α is the blood absorption coefficient given by Eq. (19). The concentration of hemoglobin c is assumed to be 15 g/100 ml, or equivalently, 2.32×10³ μmolar.

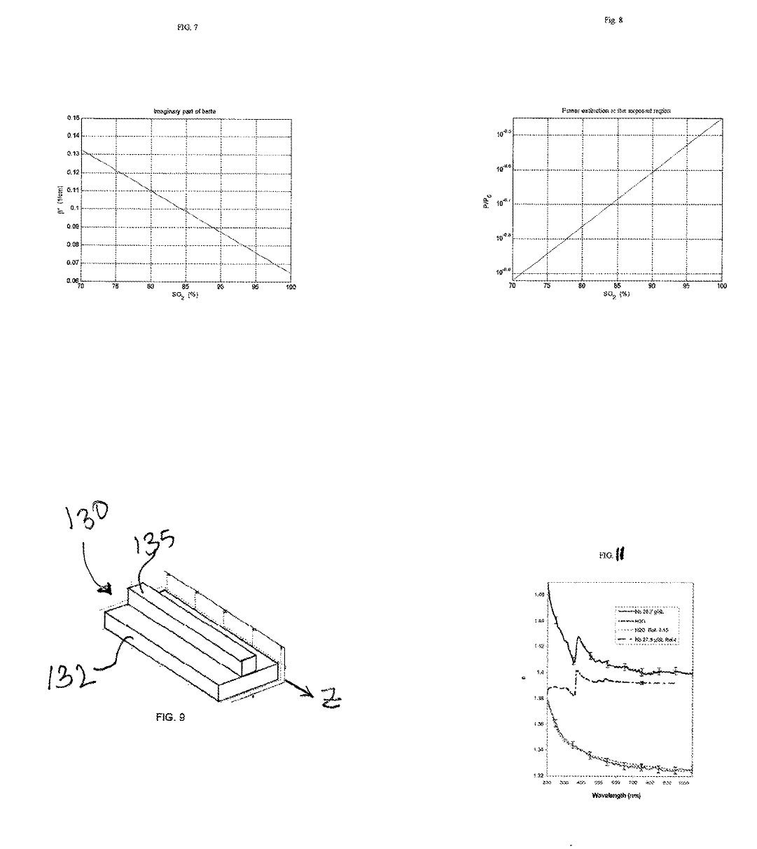

FIG. 7 shows imaginary part of β calculated from Eq. (10), Eq. (12) and Eq. (13) as a function of saturation level SO₂ that is defined as the ratio between c_{HbO}_{₂} and c_{Hb}. From this figure, we see that β″ changes linearly as the saturation level of O₂ changes from 70% to 100%. The characteristic distance L₀ can also be modeled from Eq. (16) at the corresponding minimum of β″=0.06 (100% O₂₎ saturation is 4.2 cm. Therefore, in this example an optimal length of the exposed region should be about 4 cm. FIG. 8 shows power extinction (Eq. 16) as a function of the saturation level. The extinction increases logarithmically with the saturation level.

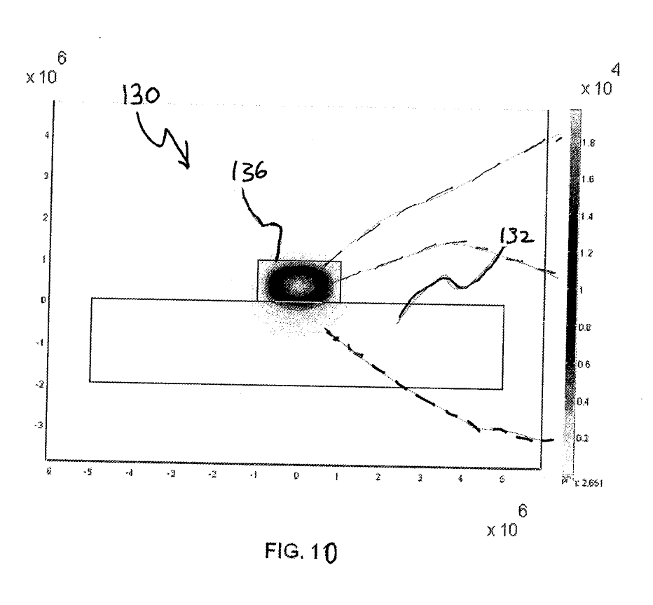

We now consider in more detail the particular advantages of the embodiment of the invention utilizing a planar dielectric waveguide 130 shown in device 100 of FIG. 6A-C. The geometry of this dielectric waveguide used for measuring oxygen saturation level of blood is shown in FIG. 9, waveguide 130 has a substrate 132 that acts as a lower cladding and a rectangular core 135. In calculating the single mode dielectric waveguide electric field distribution by finite element methods for the dielectric waveguide 130 of FIG. 9, which is plotted in FIG. 10 the refractive index of the waveguide core 135 was assumed to be n₁=1.45 while the refractive index of the substrate is 1.40 for measuring the performance at the wavelength of λ=600 nm. FIG. 10 has a shaded legend bar at the right side in which dashed lead lines connect the proper shaded portion of the bar to the corresponding shaded regions of the waveguide 130 to show the gradation in the evanescent field. The model of the device performance assumes the core region 135 of waveguide 130 is uncovered and is in the direct contact with blood. The saturation level of the blood is assumed to be 97%. Applying finite element methods to calculate mode field of the waveguide 130, shown in FIG. 11, also resulted in the imaginary part of the effective refractive index being 1.88×10⁻⁶.

Applying Eq. 16 and 17, this corresponds to the β″=0.2 cm⁻¹ and to the effective waveguide length L=1.25 cm, which is about a third less than the optimum length (4 cm) of the exposed region for a circular optical fiber. Thus, deployment of the planar waveguide permits the portion exposed to the blood to be less than 3.5 cm.

Another important advantage of the novel planar geometry sensor compared with the prior art disclosure of optical fiber sensors is the much lower potential for error in blood saturation measurement. Sources of potential errors include variations of the optical signal of the laser and inside the optical fiber and photodetector noises. This can be modeled by letting x=c_{HbO}_{₂}/c be the saturation level. Then variation of x from Eq. (19) and Eq. (21) is

$$δ x = \frac{0.43}{c Δ μ} δα \qquad ( 22 )$$

where Δμ=μ_{HbO}_{₂}−μ_{Hb} and δα is the uncertainty in the measurement the level of blood absorption coefficient. For n₂″<<1α is proportional to β″ with a good accuracy. Writing β″=Γα, where Γ is a constant for a given fiber we could rewrite Eq. (22) in the form

$$δ x = \frac{0.43}{c ΓΔ μ} {δβ}^{″} . \qquad ( 23 )$$

The error in the determination of the saturation level from the intensities I₁, I₂ is from Eq. (16) and Eq. (23)

$$δ x = 0.11 \frac{ξ}{Γ Lc Δ μ} , \qquad ( 24 )$$

where ξ=δI₁/I₁−δI₂/I₂ is the difference of intensity variations on the two photodetectors.

From Eq. (24) we see that δx becomes smaller with the increase of Δμ, L and Γ. In the equation above Δμ is determined by the wavelength of the light and Γ depends on the configuration of the fiber/waveguide.

Thus, now applying Eq. 24 to the conventional fiber optic sensor considered above, with the exposed length, L being fixed at 4 cm for maximum saturation: Γ=3.1×10⁻³, cΔμ=2.32 cm⁻¹ and δx=2.7ξ. Thus, if for example ξ=0.01, then the error in the saturation level is about 3%.

In the example of the inventive planar dielectric waveguide 130 of device 100 in FIG. 6, the exposed length, L at maximum saturation is 1.25 cm: Γ=0.019 and δx=1.4ξ. For the same level of intensity variations, the saturation error level is 1.4%, or less than half the error (3%) of the fiber optic sensor.

Thus, another aspect of the invention involves the method of first providing a waveguide comprising a planar support as a cladding on a first surface with a second surface parallel to the plane of the first surface, and terminating with a reflective surface orthogonal to the direction of propagation, then placing the second surface in contact with blood and propagating light through the waveguide toward the mirror, after which the intensity of light reflected by the mirror is measured.

Yet another important operative principle of an even more preferred embodiment of the current invention for measuring blood oxygen saturation level is to deploy a waveguide in the which the dimensions of the evanescent field is comparable to, and most preferably, much more than the dimensions of the red blood cell. It should be appreciated that if the evanescent field that interacts with the RBC is much smaller than a RBC the signal will be strongly influenced by position of a particular red blood cell relative to the waveguide. The blood component hemoglobin is concentrated within erythrocytes or red blood cells that have a torus-like shape with the diameter of each corpuscular being is about 8 μm and having a thickness of about 2 μm. Further, the evanescent field should be of a nature that allows it to also penetrate deeply into the red blood cell, that is at least a micron, or preferably at least about 2 microns, but more preferably about 4 microns. These two conditions are fulfilled for a low-dielectric-constant (low-k) planar dielectric waveguide described below.

The spatial extension of evanescent field is proportional to λ/Δn, where Δn is the difference of refractive index between core and the cladding. The refractive index of the human red blood cells is in the range 1.38-1.42 depending on concentration of the hemoglobin in it, as shown in FIG. 11. Therefore, the refractive index of the core of the waveguide should be as close to these values as possible to allow the evanescent light penetrate deeply into the red blood cell. The refractive index of SiO₂ waveguide is about 1.45. Thus, the typical penetration depth at that difference in refractive indexes between silica waveguide and red blood cell is 200-300 nm, or about between a tenth and a sixth of the thickness (2 μm) of the red blood cell. Accordingly, it is preferable that the waveguide 130 has a refractive index (n) is less than 1.45 at the absorption bands of Hb and HbO₂. It is more preferable that the refractive index of the waveguide be in the range of 1.38-1.45.

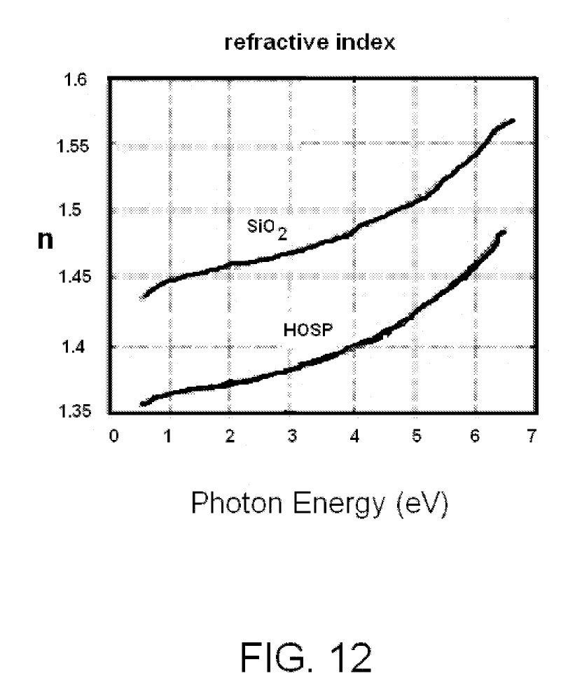

Thus, another embodiment of the invention is use of a dielectric material for a waveguide with refractive index lower than of silica to increase the penetration depth of the evanescent field, and thus obtain both a greater and more representative measurement of the blood oxygenation. One such preferred low-dielectric-constant material is spin-on hybrid siloxane-organic polymer, such as that known as HOSP and available from Honeywell Advanced Microelectronic Materials (Tempe, Ariz.). Thin films of HOSP could be prepared by a spin-on coating technique. The dispersion of refractive index of HOSP in wavelength range of 180 nm to 2.35 μm is shown in the FIG. 12.

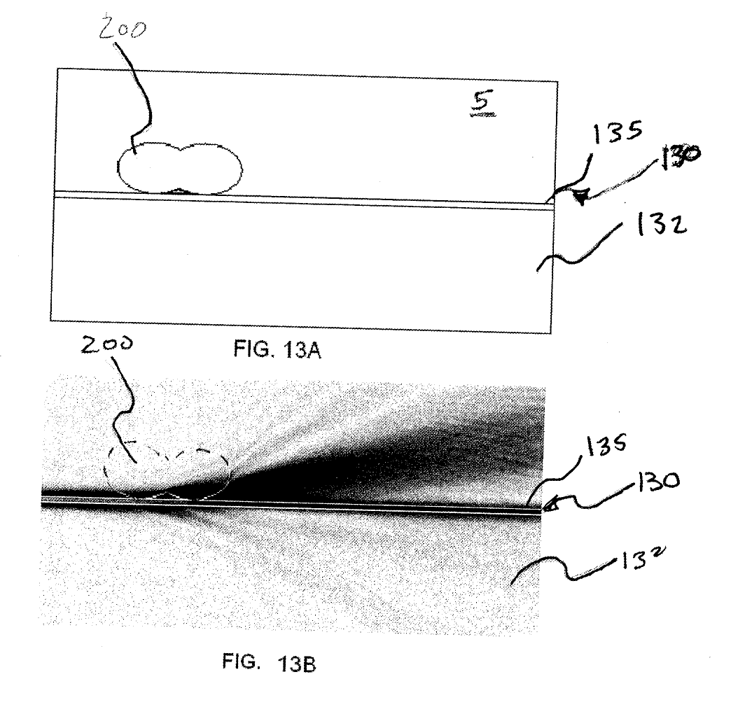

FIG. 13B illustrates the results of modeling the electric field intensity distribution for the geometry shown in FIG. 13A. The field propagates from the left to the right of the waveguide 130. The planar waveguide modeled as having been fabricated by deposition of a film of the HOSP material as the core 130 on a silicon substrate that serves as one side of the cladding. The other side of the cladding 135 of the waveguide may be formed by ion implantation into the HOSP layer. The thickness of the waveguide core is 0.5 μm. The refractive index of the core 130 of the waveguide was 1.375 and the refractive index of the cladding was 1.36. The propagation of light in the planar waveguide was then modeled as if surrounded by blood that was a solution of erythrocytes in water with a refractive index, n, of 1.33. The complex refractive index of red blood cell was modeled as 1.38+iα/2k₀, where α is the absorption coefficient. The model geometry is shown in the FIG. 13A. This figure shows only a small part of the waveguide (L=40 μm) and a single particle on it. Preferably, the waveguide 130 has a length of at least about 10 microns. Such waveguides as modeled herein will result in an accurate determination of concentrations of blood components by sampling more blood cells and sampling each red blood cell to a greater depth. Further, as the planar waveguide can have a low measurement error with a small area, such a precise measurement of oxygenated and reduced hemoglobin can be made at a particular local as the oximeter probe is inserted in a catheter or other implanted medical devices.

It should be appreciated from the foregoing that although a preferred form of a waveguide is a planar waveguide flush with the surface of the catheter or probe, the performance of an optical fiber (with a circular cross section, or any other non-planar waveguide form) is improved when the core refractive index is less about 1.45 at the absorption bands of Hb and HbO₂

In other embodiments of the invention either the planar waveguide or a non-planar waveguide using a low refractive index core may be integrated with a catheter in signal communication with cardiac monitoring equipment, or a pacemaker or electro-cardiac defibrillator to change the pacing rate or provide a defibrillating pulse when local low blood concentrations are detected so as to prevent cardiac or other tissue ischemia.

While the invention has been described in connection with a preferred embodiment, it is not intended to limit the scope of the invention to the particular form set forth, but on the contrary, it is intended to cover such alternatives, modifications, and equivalents as may be within the spirit and scope of the invention as defined by the appended claims.

1. A method of determining the concentration of oxygenated and deoxygenated hemoglobin in blood, the method comprising the steps of:
a) providing a waveguide comprising a planar support as a cladding on a first surface with a second surface parallel to the plane of the first surface, and terminating with a reflective surface orthogonal to the direction of propagation,
b) placing the second surface of the waveguide in contact with blood
c) propagating light through the waveguide toward the mirror,
d) measuring the intensity of light reflected by the mirror.

2. A method according to claim 1 wherein said waveguide has a refractive index (n) that is between about 1.38 and about 1.45 at the absorption bands of Hb and HbO₂

3. A method according to claim 1 wherein in said step of propagating light through the waveguide toward the mirror light enters the waveguide from an optical fiber.

4. A method according to claim 3 wherein in said step of measuring the intensity of the light reflected by the mirror the light propagates to the detector via an optical fiber.

5. A method according to claim 4 wherein the light propagates to and from the waveguide via the same optical fiber.

6. A method according to claim 1 wherein the evanescent light is capable of substantially penetrating into red blood cells.

7. A method according to claim 6 wherein the waveguide has a length of at least about 10 microns and less than about 1.3 cm.

8. A method according to claim 6 wherein the waveguide has a length of less than about 40 microns.

9. A device for measuring the concentration of oxygenated and deoxygenated hemoglobin by direct contact with blood which comprises:
a) waveguide comprising a planar support as a cladding on a first surface with a second surface parallel to the plane of the first surface and capable of being exposed directly to a fluid,
b) a reflective surface orthogonal to the direction of propagation of light within the waveguide,
c) means for illuminating said waveguide,
d) means for detecting the intensity of light reflected by said mirror in relationship to the intensity of light used to illuminate the waveguide.

10. A device according to claim 9 wherein the means for illuminating said waveguide is an optical fiber.

11. A device according to claim 9 wherein the waveguide has a length of at least about 10 microns and less than about 3.5 cm.

12. A device according to claim 9 wherein the light reflected by said reflective surface is transmitted to the detection means by an optical fiber.

13. A device according to claim 9 wherein the evanescent light is capable of substantially penetrating into red blood cells.

14. A device according to claim 9 wherein the waveguide has a refractive index (n) that is less than 1.45 at the absorption bands of Hb and HbO₂.

15. A device according to claim 9 wherein the waveguide has a refractive index (n) that is between about 1.375 and about 1.45 at the absorption bands of Hb and HbO₂.

16. A device according to claim 9 wherein the waveguide has a refractive index (n) that is between about 1.38 and about 1.42 at the absorption bands of Hb and HbO₂.

17. A device according to claim 15 wherein the waveguide has a refractive index (n) that is about 1.375 and the refractive index of the cladding is about 1.36 at the absorption bands of Hb and HbO₂.

18. A device according to claim 15 wherein the waveguide has a length of at least about 10 microns and less than about 40 microns.

19. A device according to claim 16 wherein the waveguide has a length of at least about 10 microns and less than about 40 microns.

20. A device according to claim 9 wherein the planar waveguide is fabricated by the deposition of a film of HOSP material as the core on a silicon substrate.

21. A device according to claim 10 wherein the light not coupled from the optical fiber into the planar waveguide is not returned to the detection means.

22. A device according to claim 10 wherein the light not coupled from the optical fiber into the planar waveguide is absorbed at the end of the optical fiber.
